(19) United States
(12) Patent Application Publication
(10) Pub. No.: US 2025/0268832 A1
(43) Pub. Date: Aug. 28, 2025

(54) Pretomanid Compositions

(71) Applicant: The Global Alliance for TB Drug Development, Inc., New York, NY (US)

(72) Inventors: Thomas Brad Gold, Greenville, NC (US); Graham Stanley Leonard, St. Albans (GB); Rajneesh Taneja, New York, NY (US)

(21) Appl. No.: 17/628,263

(22) PCT Filed: Jul. 15, 2020
(86) PCT No.: PCT/US2020/042082

§ 371 Date: Jan. 19, 2022

Related U.S. Application Data
(60) Provisional application No. 62/876,257, filed on Jul. 19, 2019.

(51) Int. Cl. A61K 9/20; A61K 9/16; A61K 31/5365

(52) U.S. Cl. CPC A61K 9/2077; A61K 9/1623; A61K 9/1635; A61K 9/1652; A61K 9/2009; A61K 9/2018; A61K 9/2027; A61K 9/2054; A61K 9/2059; A61K 31/5365

(57) ABSTRACT

Described is an oral pharmaceutical composition including a granulate including a pharmaceutically effective amount of pretomanid or pharmaceutically acceptable solvate thereof. Such granulate may have a bulk density in a range of about 0.3 to 0.8 g/mL and/or a particle size distribution such that no more than about 30 wt. % of the granulate is retained on an ASTM #60 (250 μm) sieve. In particular, the composition may provide that at least 40 wt. % of the pretomanid (e.g., at least 60 wt. %) is dissolved within 20 minutes as measured in a USP-II Apparatus at 37±2° C. in 0.5% hexadecyltrimethylammonium bromide (HDTMA) in 0.1N HCl.

### CROSS-REFERENCE TO RELATED APPLICATION(S)

This application is the United States national phase of International Application No. PCT/US2020/042082 filed Jul. 15, 2020, and claims the priority benefit of U.S. Provisional Application No. 62/876,257 filed Jul. 19, 2019, the disclosures of which are incorporated by reference herein in their entirety.

### FIELD OF THE INVENTION

The present disclosure relates to pharmaceutical compositions comprising pretomanid or a pharmaceutically acceptable solvate thereof.

### BACKGROUND

Mycobacterium tuberculosis bacteria are the causative agents of the infectious disease tuberculosis (TB), which is one of the top causes of death worldwide. According to the World Health Organization (WHO), there were an estimated 10 million people who fell ill with TB in 2017, of which 1.6 million died from the disease.

Failure to properly treat tuberculosis has caused global drug resistance in Mycobacterium tuberculosis and thus rendered typical first-line medications such as isoniazide and rifampin ineffective. According to the WHO, this remains a public health crisis and a health security threat, with approximately half a million new cases of multidrug-resistant tuberculosis occurring annually. Second-line treatment options are limited and require extensive chemotherapy (up to 2 years of treatment) with medicines that are expensive and toxic. In addition, many oral treatment regimens available to treat multidrug-resistant tuberculosis are complex and long, typically requiring a combination of at least 5 drugs administered for 18 months or more.

Pretomanid, also known as PA-824, is a nitromidazooxazine antimycobacterial agent that has recently been studied in clinical trials alone and in combination with other anti-TB drugs. Pretomanid has many attractive characteristics for use in TB therapy, including a novel mechanism of action, activity in vitro against all tested drug-resistant clinical isolates, and activity as both a potent bactericidal and sterilizing agent in mice. However, pretomanid is a poorly soluble Biopharmaceutics Classification System (BCS) Class II/IV compound. These classes of compounds are known to exhibit problematic characteristics for effective oral delivery, including low aqueous solubility, poor permeability, and poor absorption. As such, formulation and development of pharmaceutical compositions including these drugs into dosage forms, for example, immediate release dosages, is highly challenging, unpredictable, and can be driven by trial and error.

### SUMMARY

An object of certain embodiments of the present disclosure is to provide an oral pharmaceutical composition comprising a granulate comprising a pharmaceutically effective amount of pretomanid or a pharmaceutically acceptable solvate. Various aspects of the present disclosure may be further characterized by one or more of the following clauses:

Clause 1: An oral pharmaceutical composition comprising a granulate comprising a pharmaceutically effective amount of pretomanid or a pharmaceutically acceptable solvate thereof, wherein the granulate has a bulk density in a range of about 0.3 to 0.8 g/mL, wherein the granulate has a particle size distribution such that no more than about 30 wt % of the granulate is retained on an ASTM #60 (250 μm) sieve, and wherein at least 40 wt. % of the pretomanid (e.g., at least 60 wt. %) is dissolved within 20 minutes as measured in a USP-II Apparatus at 37÷2° C. in 0.5% hexadecyltrimethylammonium bromide (HDTMA) in 0.1N HCl.

Clause 2: The pharmaceutical composition of clause 1, wherein the bulk density range is about 0.47 to about 0.53 g/mL.

Clause 3: The pharmaceutical composition of clause 1 or 2, wherein the particle size distribution is such that about 5 wt. % to about 30 wt. % of the granulate is retained on the ASTM #60 (250 μm) sieve.

Clause 4: The pharmaceutical composition of any one of claims 1 to 3, wherein the particle size distribution is such that at least 80 wt. % of the granulate is retained on an ASTM #200 (75 μm) sieve.

Clause 5: The pharmaceutical composition of any one of clauses 1 to 4, wherein the composition has a disintegration time of less than 10 minutes

Clause 6: The pharmaceutical composition of any one of clauses 1 to 5, wherein at least 60 wt. % of the pretomanid is dissolved within 10 minutes in 0.5% HDTMA in 0.1N HCl.

Clause 7: The pharmaceutical composition of any one of clauses 1 to 6, wherein at least 75 wt. % of the pretomanid is dissolved within 40 minutes in 0.5% HDTMA in 0.1N HCl.

Clause 8: The pharmaceutical composition of any one of clauses 1 to 7, wherein the composition has a disintegration time of less than or equal to 5 minutes.

Clause 9: The pharmaceutical composition of any one of clauses 1 to 8, wherein the pretomanid comprises at least 10 wt. % (e.g., from 10 wt. % to 50 wt. % or 10 wt. % to 30 wt. %) of the pharmaceutical composition.

Clause 10: The pharmaceutical composition of any one of clauses 1 to 9, comprising the granulate and one or more pharmaceutically acceptable extragranular excipients, wherein the granulate comprises the pretomanid and one or more pharmaceutically acceptable intragranular excipients.

Clause 11: The pharmaceutical composition of clause 10, wherein each excipient is independently selected from the group consisting of a diluent, a disintegrant, a binder, a surfactant, a glidant, and a lubricant.

Clause 12: The pharmaceutical composition of clause 10 or 11, wherein the one or more intragranular excipients comprise a diluent, a disintegrant, a binder, and a surfactant.

Clause 13: The composition of clause 11 or 12, wherein each diluent is selected from the group consisting of a saccharide, a disaccharide, a sugar alcohol, a polysaccharide, and a polysaccharide derivative.

Clause 14: The composition of any one of clauses 11 to 13, wherein each diluent is a disaccharide or a polysaccharide.

Clause 15: The composition of any one of clauses 11 to 14, wherein the diluent comprises lactose monohydrate (e.g. spray-dried lactose monohydrate) and microcrystalline cellulose.

Clause 16: The composition of any one of clauses 11 to 15, wherein the binder comprises a polymeric binder.

Clause 17: The composition of any one of clauses 11 to 16, wherein the binder comprises polyvinylpyrrolidone.

Clause 18: The composition of any one of clauses 11 to 17, wherein the surfactant comprises sodium lauryl sulfate, ammonium lauryl sulfate, docusate sodium, ammonium dinonyl sulfosuccinate, diamyl sulfosuccinate sodium, dicapryl sulfosuccinate sodium, diheptyl sulfosuccinate sodium, dihexyl sulfosuccinate sodium, diisobutyl sulfosuccinate sodium, ditridecyl sulfosuccinate sodium, sodium dodecylbenzenesulfonate, or a mixture thereof.

Clause 19: The composition of any one of clauses 11 to 17, wherein the surfactant comprises an alkylphenol, a fatty acid glyceride, a sorbitan ester, an ethoxylated fatty acid, an ethoxylated fatty alcohol, an ethoxylated alkylphenol, an ethoxylated hydrogenated vegetable oil, an ethoxylated sorbitan ester, an ethoxylated fatty acid amides, or a mixture thereof.

Clause 20: The composition of any one of clauses 11 to 19, wherein the surfactant comprises sodium lauryl sulfate.

Clause 21: The composition of any one of clauses 11 to 20, wherein the disintegrant comprises low substituted hydroxypropyl cellulose, carboxymethylcellulose sodium, croscarmellose sodium, crospovidone, and sodium starch glycolate, or a mixture thereof.

Clause 22: The composition of any one of clauses 11 to 21, wherein the disintegrant comprises sodium starch glycolate.

Clause 23: The composition of clause 10 or 11, wherein the extragranular excipients comprise a lubricant, a disintegrant, and a glidant.

Clause 24: The composition of clause 11 or 23, wherein the lubricant comprises lauric acid, myristic acid, palmitic acid, stearic acid or pharmaceutically acceptable salts or esters thereof, such as magnesium stearate, calcium stearate, sodium stearyl fumarate, or zinc stearate or a mixture thereof

Clause 25: The composition of any one of clauses 11, 23, or 24, wherein the lubricant comprises magnesium stearate.

Clause 26: The composition of any one of clauses 23 to 25, wherein the disintegrant comprises low substituted hydroxypropyl cellulose, carboxymethylcellulose sodium, croscarmellose sodium, crospovidone, and sodium starch glycolate, or a mixture thereof.

Clause 27: The composition any one of clauses 23 to 26, wherein the disintegrant comprises sodium starch glycolate.

Clause 28: The composition of any one of clauses 11 and 23 to 27, wherein the glidant comprises talc, calcium phosphate, calcium silicate, magnesium silicate, magnesium trisilicate, silicon dioxide, colloidal silicon dioxide, magnesium aluminosilicate, or a mixture thereof.

Clause 29: The composition of any one of clauses 11 and 23 to 28, wherein the glidant comprises colloidal silicon dioxide.

Clause 30: The pharmaceutical composition of any one of clauses 11 to 29, wherein the composition comprises: from 10 wt. % to 30 wt. % of the pretomanid, from 60 wt. % to 85 wt. % of the diluent, from 1 wt. % to 10 wt. % of the disintegrant, from 0.1 wt. % to 1 wt. % of the surfactant, from 1 wt. % to 5 wt. % of the binder, from 0.1 wt. % to 1 wt. % of the glidant, and from 0.1 wt. % to 3 wt. % of the lubricant.

Clause 31: The pharmaceutical composition of any one of clauses 1 to 30, wherein the composition comprises from 50 mg to 250 mg of the pretomanid.

Clause 32: The pharmaceutical composition of any one of clauses 1 to 31, wherein the composition comprises 200 mg of the pretomanid.

Clause 33: The pharmaceutical composition of any one of clauses 1 to 32, wherein the composition comprises 100 mg of the pretomanid.

Clause 34: The pharmaceutical composition of any one of clauses 1 to 33, wherein the composition is in the form of a tablet.

Clause 35: The pharmaceutical composition of clause 34, wherein the tablet has a hardness within the range of 7 to 13 Kp.

Clause 36: A method for treating tuberculosis comprising, administering to a patient in need of such treatment, a pharmaceutical composition of any one of clauses 1 to 35.

Clause 37: The method of clause 36, further comprising co-administering to the patient one or more separate dosage forms comprising therapeutically effective amounts of additional active pharmaceutical ingredients (APIs) selected from the group consisting of bedaquiline, moxifloxacin, linezolid, pyrazinamide, and pharmaceutically acceptable salts or solvates of the preceding.

Clause 38: The method of clause 37, comprising co-administering to the patient one or more bedaquiline fumarate oral tablets and one or more linezolid oral tablets.

Clause 39: The method of clause 38, comprising co-administering to the patient one or more bedaquiline fumarate oral tablets, one or more moxifloxacin hydrochloride oral tablets, and one or more pyrazinamide oral tablets.

Clause 40: The method of any one of clauses 36 to 39, wherein the tuberculosis is drug-susceptible tuberculosis (DS-TB).

Clause 41: The method of any one of clauses 36 to 39 wherein the tuberculosis is multi-drug resistant tuberculosis (MDR-TB).

Clause 42: The method of any one of clauses 36 to 39, wherein the tuberculosis is extensively-drug resistant tuberculosis (XDR-TB).

Clause 43: A process for preparing an oral pharmaceutical composition comprising: preparing a granulate comprising a pharmaceutically effective amount of pretomanid and one or more pharmaceutically acceptable intragranular excipients, and combining the granulate with one or more pharmaceutically acceptable extragranular excipients to provide a blend.

Clause 44: The process of clause 43, further comprising compressing at least a portion of the blend to provide a solid dosage.

Clause 45: The process of clause 43 or 44, wherein the granulate is prepared by mixing a mixture of pretomanid and the one or more pharmaceutically acceptable intragranular excipients, and subsequently granulating the mixture.

Clause 46: The process of any one of clauses 43 to 45, wherein granulating the mixture comprises a wet granulation method.

Clause 47: The process of any one of clauses 43 to 45, wherein granulating the mixture comprises a dry granulation method.

Clause 48: The process of any one of clauses 43 to 45, wherein granulating the mixture comprises a melt granulation method.

Clause 49: The process of any one of clauses 43 to 48, wherein each excipient is independently selected from the group consisting of a diluent, a disintegrant, a binder, a surfactant, a glidant, and a lubricant.

Clause 50: The process of any one of clauses 43 to 49, wherein the one or more intragranular excipients comprise a diluent, a disintegrant, a binder, and a surfactant.

Clause 51: The process of any one of clauses 43 to 50, wherein the one or more extragranular excipients comprise a lubricant, a disintegrant, and a glidant.

Clause 52: The process of any one of clauses 45 to 51, wherein mixing the mixture of pretomanid and the one or more pharmaceutically acceptable intragranular excipients comprises dry mixing pretomanid and one or more of the intragranular excipients to form a dry mixture and adding to the dry mixture a binder solution.

Clause 53: The process of clause 52, wherein the binder solution comprises a binder, a surfactant, and a solvent.

Clause 54: The process of any one of clauses 43 to 53, wherein the granulate has a bulk density in a range of about 0.3 to 0.8 g/mL.

Clause 55: The process of any one of clauses 43 to 54, wherein the granulate has a particle size distribution such that no more than about 30 wt % of the granulate is retained on an ASTM #60 (250 μm) sieve.

Clause 56: The process of any one of clauses 43 to 55, wherein the particle size distribution of the granulate is such that about 5 wt. % to about 30 wt. % of the granulate is retained on the ASTM #60 (250 μm) sieve.

Clause 57: The process of any one of clauses 43 to 56, wherein the particle size distribution of the granulate is such that at least 80 wt. % of the composition is retained on an ASTM #200 (75 μm) sieve.

Clause 58: A process for preparing an oral pharmaceutical composition comprising: preparing a blend comprising a pharmaceutically effective amount of pretomanid and one or more pharmaceutically acceptable excipients, and compressing at least a portion of the blend to provide a solid dosage.

Clause 59: The process of claim 58, wherein each excipient is independently selected from the group consisting of a diluent, a disintegrant, a binder, a surfactant, a glidant, and a lubricant.

Clause 60: An oral pharmaceutical composition prepared according to the process of any one of clauses 43 to 59.

Clause 61: An oral pharmaceutical composition comprising a granulate comprising a pharmaceutically effective amount of pretomanid or a pharmaceutically acceptable solvate thereof, wherein at least 70 wt. % of the pretomanid is dissolved within 10 minutes as measured in a USP-II Apparatus at 37±2° C. in 0.5% hexadecyltrimethylammonium bromide (HDTMA) in 0.1N HCl, the granulate has a bulk density between about 0.47 and 0.53 g/mL; and the granulate has a particle size distribution such that between about 5 wt. % and about 30 wt. % of the granulate is retained on an ASTM #60 (250 μm) sieve.

### DETAILED DESCRIPTION

For purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances. Further, in this application, the use of “a” or “an” means “at least one” unless specifically stated otherwise. For example, “a” diluent, “an” intragranular excipient, “a” disintegrant, and the like refer to one or more of any of these items.

The proportions of each component of the pharmaceutical composition used herein are defined as “% wt.”, i.e. weight percent, which is calculated based on the total weight of the pharmaceutical composition.

“About” as used herein means ±10% of the referenced value. In certain embodiments, “about” means ±9%, or ±8%, or ±7%, or ±6%, or ±5%, or ±4%, or ±3%, or ±2% or ±1% of the referenced value.

The present disclosure is directed to a pharmaceutical composition comprising a granulate comprising a pharmaceutically effective amount of pretomanid or a pharmaceutically acceptable solvate thereof. The pharmaceutical composition can be formulated into several dosage forms, including but not limited to, solid dosage forms for oral administration such as capsules, tablets, pills, powders, and granules.

Pretomanid is a nitroimidazole antibacterial drug that is active against Mycobaterium tuberculosis. Pretomanid is specifically a nitroimidazooxazine having the IUPAC chemical name (65)-2-nitro-6-{[4-(trifluoromethoxy)benzyl]oxy}-6,7,-dihydro-5H-imidazol[2.1b][1.3]oxazine. The structure of Pretomanid is depicted below in Formula I:

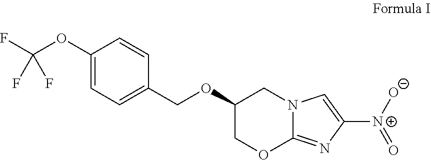 [SMILES: O=[N+]([O-])c1cn2c(n1)OC[C@@H](OCc1ccc(OC(F)(F)F)cc1)C2]

Pretomanid is classified as a poorly soluble Class II/IV compound of the Biopharmaceutics Classification System (BCS). These classes of compounds are known to exhibit problematic characteristics for effective oral delivery, including low aqueous solubility, poor permeability, and poor absorption. As such, formulation and development of dissolution methods for these classes of compounds is highly challenging and unpredictable. It has been found that the pharmaceutical composition of the present invention, including one or more pharmaceutical excipients as described below, allows for an efficacious release composition of this BCS Class II/IV compound.

The pretomanid can comprise at least 1 wt. %, at least 5 wt. %, or at least 10 wt. % of the pharmaceutical composition. The pretomanid can comprise up to 50 wt. %, up to 40 wt. %, or up to 30 wt. % of the pharmaceutical composition. For example, the pharmaceutical composition can comprise pretomanid within a range such as from 1 wt. % to 50 wt. %, from 5 wt. % to 40 wt. %, or from 10 wt. % to 30 wt. %, such as 25 wt. % or 12.5 wt. %.

“Pharmaceutically acceptable salts” can include, without limitation, salts derived from an appropriate base, such as an alkali metal (for example, sodium), an alkaline earth (for example, magnesium), ammonium and NR₄⁺ (wherein R is C₁-C₁₀ alkyl), an organic acid such as fumaric acid, acetic acid, succinic acid, or an inorganic acid such as hydrochloric acid or hydrobromic acid. Physiologically acceptable salts of a hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound of a hydroxy or carboxy group include the anion of said compound in combination with a suitable cation such as sodium, potassium, calcium, magnesium, lithium, and zinc, and NR₄⁺ (wherein R is independently selected from H or a C₁-C₁₀ alkyl group). “Alkyl” as used herein refers to a univalent group derived from a linear or branched alkane by removal of a hydrogen atom from any carbon atom (—CₙH2ₙ₊₁). Examples of alkyl groups include, but are not limited to, methyl, ethyl, isopropyl, sec-butyl, n-butyl, hexyl, octyl, and decyl.

“Solvate” as used herein refers to a complex of variable stoichiometry formed by a solute (the referenced compound) and a solvent. Solvents, by way of example, include water (such can be referred to as a “hydrate”), methanol, ethanol, and acetic acid. The phrase “salt and/or solvate” refers to each of a salt (e.g., moxifloxacin hydrochloride), a solvate, and a solvate of a salt (e.g., moxifloxacin hydrochloride monohydrate).

The pharmaceutical composition of the present disclosure can include one or more pharmaceutically acceptable excipients. Suitable excipients may include, but are not limited to, one or more: diluents, binders (e.g., polymeric binders), humectants, disintegrating agents, solution retarders, absorption accelerators, wetting agents, adsorbents, lubricants, surface stabilizers (surfactants), viscosity building agents, glidants, buffering agents, and flavoring agents. The one or more pharmaceutically acceptable excipients may be intragranular and/or extragranular excipients.

The one or more excipients can comprise at least 25 wt. %, at least 50 wt. %, at least 60 wt. %, or at least 75 wt. % of the pharmaceutical composition. The one or more excipients can comprise up to 99 wt. %, up to 95 wt. %, or up to 90 wt. % of the pharmaceutical composition. For example, the pharmaceutical composition can comprise the one or more excipients within a range such as from 50 wt. % to 99 wt. %, from 60 wt. % to 95 wt. %, or from 75 wt. % to 90 wt. %.

Suitable pharmaceutically acceptable diluents, also known as solid carriers or fillers, may include one or more saccharides, disaccharides, and sugar alcohols such as lactose (for example, spray-dried lactose, α-lactose, and β-lactose, such as spray-dried lactose monohydrate available under the trade mark SuperTab® (available from DFE Pharma, Goch, Germany), lactitol, sucrose, sorbitol, mannitol, dextrose; polysaccharide and polysaccharide derivatives such as dextrates, dextrin, maltodextrin, dextran, croscarmellose sodium, microcrystalline cellulose (for example, microcrystalline cellulose available under the trade mark AVICEL, FMC Corp., Philadelphia, Pennsylvania; such as Avicel® PH-102 having a particle size distribution of D10, 15-55 μm, D50 80-140 μm, and D90, 170-283 μm), hydroxypropylcellulose, low-substituted hydroxypropylcellulose, hydroxypropyl methylcellulose (HPMC), methylcellulose polymers (such as, for example, METHOCEL A, METHOCEL A4C, METHOCEL A15C, METHOCEL A4M; Dow Chemical, Midland, Michigan), hydroxyethylcellulose, sodium carboxymethylcellulose, carboxymethyl hydroxyethylcellulose, starches (including potato starch, corn starch, maize starch and rice starch), and modified starches; and mixtures thereof.

In one embodiment, each diluent is independently selected from the group consisting of an inorganic compound, a saccharide, a disaccharide, a sugar alcohol, a polysaccharide, and a polysaccharide derivative. Suitable inorganic compounds may include but are not limited to calcium carbonate, dibasic calcium phosphate, tribasic calcium phosphate, calcium sulfate, magnesium carbonate, magnesium oxide, sodium chloride, and talc. In one embodiment, each diluent is independently selected from the group consisting of a saccharide, a disaccharide, a sugar alcohol, a polysaccharide, and a polysaccharide derivative. In one embodiment, the diluent comprises a saccharide, a disaccharide, or a sugar alcohol; and a polysaccharide or polysaccharide derivative. In another embodiment, the diluent comprises a disaccharide and a polysaccharide. In another embodiment, the diluent comprises lactose, lactitol, sucrose, sorbitol, mannitol, dextrin, dextrose, maltodextrin, microcrystalline cellulose, starch, or a mixture thereof. In another embodiment, the diluent comprises a saccharide, a disaccharide, or a sugar alcohol; and microcrystalline cellulose. In another embodiment, the diluent comprises a disaccharide and microcrystalline cellulose. In another embodiment, the diluent comprises lactose, such as lactose monohydrate, and microcrystalline cellulose. In any of the preceding embodiments, the lactose monohydrate can be SuperTab® 11SD, a spray-dried lactose having a particle size distribution of D10 about 50 μm, D50 about 120 μm; and D90 about 220 μm.

The diluent can comprise at least 25 wt. %, at least 50 wt. %, or at least 60 wt. % of the pharmaceutical composition. The diluent can comprise up to 90 wt. %, up to 85 wt. %, or up to 80 wt. % of the pharmaceutical composition. For example, the pharmaceutical composition can comprise the diluent within a range such as from 25 wt. % to 90 wt. %, from 50 wt. % to 85 wt. %, or from 60 wt. % to 85 wt. %.

Suitable binders may comprise (e.g., polymeric binders), for example, polyvinylpyrollidone (povidone, such as Kollidon® 30, BASF; or PVP K-30, having a Mw, 40 kDa-80 kDa, available from Ashland Specialty, Covington, Kentucky); polyethylene glycol(s), polyethylene oxide, cellulose derivatives including ethyl cellulose, methyl cellulose, hydroxypropylcellulose, hydroxymethylcellulose, hydroxypropylmethylcellulose, hydroxyethylcellulose, sodium carboxymethylcellulose, carboxymethyl hydroxyethylcellulose; modified starch derivatives such as hydroxypropyl starch or pregelatinized hydroxypropyl starch; polysaccharides including, starch and starch-based polymers e.g. pre-gelatinized starch; chitosan, alginates; maltodextrin; polysaccharide gums such as, but not limited to, acacia, locust bean gum, agar, dextrin, carrageenan, calcium carrageenan, casein, zein, alginic acid, sodium alginate, pectin, gelatin, xanthan gum, guar gum, fenugreek gum, gum arabic, galactomannans, gellan, konjac, inulin, karaya gum, gum tragacanth, and combinations thereof; and carbomer homopolymers of acrylic acid, or carbomer polymers of acrylic acid crosslinked with an allyl ether of pentaerythritol, sucrose, or propylene glycol. Binders may also include inorganic binders such as bentonite or magnesium aluminum silicate.

In one embodiment, the binder comprises a hydrophilic polymer. In another embodiment, the binder may comprise one or more hydrophilic polymers selected from the group consisting of polyvinylpyrrolidone (povidone); polyethylene glycol, hydroxypropylcellulose, hydroxymethylcellulose, hydroxypropylmethylcellulose, hydroxyethylcellulose, sodium carboxymethylcellulose, carboxymethyl hydroxyethylcellulose; hydroxypropyl starch or pregelatinized hydroxypropyl starch; pregelatinized starch; carbomer homopolymers, and Crosslinked Carbomer polymers. In another embodiment, the binder may comprise one or more hydrophilic polymers selected from the group consisting of polyvinylpyrrolidone (povidone); polyethylene glycol, hydroxypropylcellulose, hydroxypropylmethylcellulose, hydroxypropyl starch or pregelatinized hydroxypropyl starch; and pregelatinized starch. In another embodiment, the binder may comprise one or more hydrophilic polymers selected from the group consisting of polyvinylpyrrolidone (povidone); and hydroxypropylmethylcellulose. In another embodiment, the polymeric binder comprises polyvinylpyrrolidione (povidone).

The binder can comprise at least 0.1 wt. %, at least 0.5 wt. %, or at least 1 wt. % of the pharmaceutical composition. The binder can comprise up to 10 wt. %, up to 5 wt. %, or up to 3 wt. % of the pharmaceutical composition. For example, the pharmaceutical composition can comprise the binder within a range such as from 0.1 wt. % to 10 wt. %, from 0.5 wt. % to 5 wt. %, or from 1 wt. % to 5 wt. %.

Suitable disintegrating agents (or “disintegrants”) include, for example, hydroxypropyl cellulose (HPC), low substituted HPC (having a hydroxypropyl content of less than 15%, such as 8% or 11%), carboxymethylcellulose (CMC), sodium CMC, calcium CMC, crystalline cellulose, croscarmellose sodium, carboxymethyl starch, hydroxypropyl starch, alginic acid or a salt thereof such as sodium alginate, corn starch, potato starch, maize starch, modified starches, microcrystalline cellulose, crospovidone, sodium starch glycolate, and mixtures thereof.

In one embodiment, the disintegrant may comprise one or more disintegrants selected from the group consisting of low substituted hydroxypropyl cellulose, carboxymethylcellulose sodium, croscarmellose sodium, crospovidone, and sodium starch glycolate. In another embodiment, the disintegrant comprises sodium starch glycolate.

The disintegrant can comprise at least 0.1 wt. %, at least 1 wt. %, or at least 3 wt. % of the pharmaceutical composition. The disintegrant can comprise up to 15 wt. %, up to 10 wt. %, or up to 7 wt. % of the pharmaceutical composition. For example, the pharmaceutical composition can comprise the disintegrant within a range such as from 0.1 wt. % to 15 wt. %, from 1 wt. % to 10 wt. %, or from 3 wt. % to 7 wt. %.

Suitable surfactants may include amphoteric, non-ionic, cationic, or anionic surfactants. Examples of amphoteric surfactants include, but are not limited to, lecithin, cocamidopropyl betaine, lauryldimethylamine oxide, myristamine oxide, coco amino propionate, sodium lauryl imino dipropionate, sodium octyl imino dipropionate, sodium coco imino mono/dipropionate, oleyldimethylbetaine, and sodium N-cocoamidethyl N-hydroxyethylglycine, and mixtures thereof.

Examples of non-ionic surfactants include, but are not limited to, alkylphenols, such as 4-(2,4-dimethylheptan-3-yl)phenol; fatty acid glycerides such as glyceryl dibehenate, glyceryl monocaprylate, glyceryl monocaprylocaprate, glyceryl monostearate, and glyceryl tristearate; sorbitan esters such as sorbitan monolaurate, sorbitan monooleate, sorbitan monostearate, sorbitan sesqueoleate, sorbitan trioleate, and sorbitan tristearate; ethoxylated fatty acids such as stearic acid ethoxylate and lauric acid ethoxylate; ethoxylated fatty alcohols such as polyoxyethylene lauryl ethers (Brij); ethoxylated alkylphenols such as nonylphenol ethoxylate and ethoxylated p-tert-octylphenol; ethoxylated hydrogenated vegetable oils such as polyoxyl 35 castor oil (Cremophor EL) and hydrogenated polyoxyl 40 castor oil (Cremophor RH 40); ethoxylated sorbitan esters such as polyoxyethylene sorbitan monolaurate (polysorbate 20), polyoxyethylene sorbitan monopalmitate (polysorbate 40), polyoxyethylene sorbitan monostearate (polysorbate 60), and polyoxyethylene sorbitan monooleate (polysorbate 80); ethoxylated fatty acid amides such as cocoamide monoethanolamine and cocamide diethanolamine; and mixtures thereof.

Examples of cationic surfactants include, but are not limited to, quaternary ammonium salts such as cetyl trimethyl ammonium bromide (CTAB), methylbenzethonium chloride, and hexadecyltrimethylammonium bromide; 2-alkyl-1-hydroxyethyl-2-imidazolines such as lauryl hydroxyethyl imidazoline and stearyl hydroxyethyl imidazoline; N,N,N,N-tetrakis substituted ethylenediamines such as ethylenediamine tetrakis (ethoxylate-block-propoxylate) tetrol and ethylenediamine tetrakis (propoxylate-block-ethoxylate) tetrol; fatty amine ethoxylates such as stearyl amine ethoxylate, oleyl amine ethoxylate, tallow amine ethoxylate and coco amine ethoxylate; and mixtures thereof.

Examples of anionic surfactants include, but are not limited to, alkyl sulfates, such as sodium dodecyl sulfate (sodium lauryl sulfate) and ammonium lauryl sulfate; bile salts such as sodium deoxycholate and sodium cholate; sulfosuccinate diesters such as docusate sodium, ammonium dinonyl sulfosuccinate, diamyl sulfosuccinate sodium, dicapryl sulfosuccinate sodium, diheptyl sulfosuccinate sodium, dihexyl sulfosuccinate sodium, diisobutyl sulfosuccinate sodium, and ditridecyl sulfosuccinate sodium; alkylbenzene sulfonates such as sodium dodecylbenzenesulfonate; sodium petroleum sulfonates such as those available under the tradename PETRONATE from Sonneborn, LLC Petrolia, Pennsylvania; sodium alkyl naphthalene sulfonate such as those available under the tradename NAXAN from Nease Performance Chemicals, West Chester Township, Ohio; sulfated natural oils and glycerides such as lauryl monoglyceryl sulfate and sulfated castor oil; and sulfated ethoxylated fatty alcohols such as sodium laureth sulfate and sodium myreth sulfate; and mixtures thereof.

In certain embodiments, the surfactants may include an anionic surfactant. In one embodiment, the surfactant comprises sodium lauryl sulfate, ammonium lauryl sulfate, docusate sodium, ammonium dinonyl sulfosuccinate, diamyl sulfosuccinate sodium, dicapryl sulfosuccinate sodium, diheptyl sulfosuccinate sodium, dihexyl sulfosuccinate sodium, diisobutyl sulfosuccinate sodium, ditridecyl sulfosuccinate sodium, sodium dodecylbenzenesulfonate, or a mixture thereof. In another embodiment, the surfactant comprises sodium lauryl sulfate.

The surfactant can comprise at least 0.05 wt. %, at least 0.1 wt. %, or at least 0.2 wt. % of the pharmaceutical composition. The surfactant can comprise up to 5 wt. %, up to 2 wt. %, or up to 1 wt. % of the pharmaceutical composition. For example, the pharmaceutical composition can comprise the surfactant within a range such as from 0.05 wt. % to 5 wt. %, from 0.1 wt. % to 5 wt. %, or from 0.1 wt. % to 1 wt. %.

Suitable glidants may comprise one or more of, but not limited to talc; powdered cellulose; calcium phosphate (e.g., tribasic calcium phosphate); magnesium oxide; sodium stearate; silicic acid or a derivative or salt thereof (for example, silicates, calcium silicate, magnesium silicate, magnesium trisilicate, silicon dioxide, colloidal silicon dioxide, hydrophobic silicon dioxide, and polymers thereof); magnesium aluminosilicate (such as NEUSILIN, available from Fuji Chem. Indus. Co. Ltd., Japan); magnesium alumino metasilicate; and mixtures thereof.

In one embodiment, the glidant comprises talc, calcium phosphate, calcium silicate, magnesium silicate, magnesium trisilicate, silicon dioxide, colloidal silicon dioxide, magnesium aluminosilicate, or a mixture thereof. In another embodiment, the glidant comprises talc, calcium phosphate, silicon dioxide, colloidal silicon dioxide, or a mixture thereof. In another embodiment, the glidant comprises colloidal silicon dioxide.

The glidant can comprise at least 0.05 wt. %, at least 0.1 wt. %, or at least 0.2 wt. % of the pharmaceutical composition. The glidant can comprise up to 5 wt. %, up to 3 wt. %, up to 2 wt. %, or up to 1 wt. % of the pharmaceutical composition. For example, the pharmaceutical composition can comprise the glidant within a range such as from 0.05 wt. % to 5 wt. %, from 0.1 wt. % to 3 wt. %, or from 0.1 wt. % to 1 wt. %.

Suitable lubricants may comprise one or more of, but not limited to fatty acids such as lauric acid, myristic acid, palmitic acid, and stearic acid and pharmaceutically acceptable salts or esters thereof (for example, magnesium stearate, calcium stearate, sodium stearyl fumarate, zinc stearate or other metallic stearate); talc; polyethylene glycols (PEGs); light mineral oil; poloxamers, such as KOLLIPHOR P188 and P407 available from BASF, Ludwigshafen, Germany; polysorbates such as polysorbate 20, polysorbate 40, polysorbate 60, and polysorbate 80; sodium lauryl sulfate; sorbitan esters such as sorbitan monolaurate, sorbitan monooleate, sorbitan monopalmitate, sorbitan monostearate, sorbitan sesquioleate and sorbitan trioleate; ethoxylated fatty acids such as polyoxyl 40 stearate and polyoxyl 15 hydroxystearate; ethoxylated alkanols such as polyoxyl 20 cetostearyl ether and polyoxyl 10 oleyl ether; ethoxylated vegetable oils and ethoxylated hydrogenated vegetable oils such as polyoxyl 35 castor oil (Cremophor EL) and hydrogenated polyoxyl 40 castor oil (Cremophor RH 40); waxes (for example, microcrystalline waxes); glycerides, such as glyceryl dibehenate, glyceryl monocaprylate, glyceryl monocaprylocaprate, glyceryl monostearate, and glyceryl tristearate; sucrose ester of fatty acids such as sucrose stearate; hydrogenated vegetable oils (for example, hydrogenated castor oil and hydrogenated palm oil); and mixtures thereof.

In one embodiment, the lubricant comprises lauric acid, myristic acid, palmitic acid, stearic acid or pharmaceutically acceptable salts or esters thereof, such as magnesium stearate, calcium stearate, sodium stearyl fumarate, or zinc stearate or a mixture thereof. In another embodiment, the lubricant comprises magnesium stearate, calcium stearate, sodium stearyl fumarate, zinc stearate or a mixture thereof. In one embodiment, the lubricant comprises magnesium stearate.

The lubricant can comprise at least 0.05 wt. %, at least 0.1 wt. %, or at least 0.5 wt. % of the pharmaceutical composition. The lubricant can comprise up to 5 wt. %, up to 3 wt. %, or up to 2 wt. % of the pharmaceutical composition. For example, the pharmaceutical composition can comprise the lubricant within a range such as from 0.05 wt. % to 5 wt. %, from 0.1 wt. % to 3 wt. %, or from 0.2 wt. % to 2 wt. %.

In one embodiment, the pharmaceutical composition comprises pretomanid and one or more pharmaceutically acceptable excipients that are independently selected from the group consisting of a diluent, a disintegrant, a surfactant, a binder, a glidant, a lubricant, and a mixture thereof.

In another embodiment, the pharmaceutical composition comprises pretomanid and one or more pharmaceutically acceptable excipients that are a diluent, a disintegrant, a surfactant, a binder, a glidant, and a lubricant.

In one particular embodiment, the pharmaceutical composition comprises (i) a granulate comprising pretomanid and one or more pharmaceutically acceptable intragranular excipients and (ii) one or more pharmaceutically acceptable extragranular excipients.

The granulate can comprise at least 75 wt. %, at least 90 wt. %, or at least 95 wt. % of the pharmaceutical composition. The granulate can comprise up to 99 wt. %, up to 98 wt. %, or up to 97 wt. % of the pharmaceutical composition. For example, the pharmaceutical composition can comprise the granulate within a range such as from 75 wt. % to 99 wt. %, from 90 wt. % to 98 wt. %, or from 95 wt. % to 97 wt. %.

The one or more extragranular excipients can comprise at least 1 wt. %, at least 2 wt. %, or at least 3 wt. % of the pharmaceutical composition. The one or more extragranular excipients can comprise up to 25 wt. %, up to 10 wt. %, or up to 5 wt. % of the pharmaceutical composition. For example, the pharmaceutical composition can comprise the one or more extragranular excipients within a range such as from 1 wt. % to 25 wt. %, from 2 wt. % to 10 wt. %, or from 3 wt. % to 5 wt. %.

In one embodiment, the one or more intragranular excipients are independently selected from the group consisting of a diluent, a disintegrant, a binder, a surfactant, and mixtures thereof. In another embodiment, the one or more intragranular excipients are a diluent, a disintegrant, a binder, and a surfactant.

In another embodiment, the one or more intragranular excipients comprise a diluent comprising a saccharide, a disaccharide, or a sugar alcohol, and a polysaccharide or polysaccharide derivative; a disintegrant selected from the group consisting of low substituted hydroxypropyl cellulose, carboxymethylcellulose sodium, croscarmellose sodium, crospovidone, and sodium starch glycolate and mixtures thereof; a polymeric binder selected from the group consisting of polyvinylpyrrolidone, polyethylene glycol, hydroxypropylcellulose, hydroxymethylcellulose, hydroxypropylmethylcellulose, hydroxyethylcellulose, sodium carboxymethylcellulose, carboxymethyl hydroxyethylcellulose, hydroxypropyl starch, pregelatinized hydroxypropyl starch, pregelatinized starch, Carbomer homopolymers, crosslinked Carbomer polymers, and mixtures thereof; and a surfactant selected from the group consisting of sodium lauryl sulfate, ammonium lauryl sulfate, docusate sodium, ammonium dinonyl sulfosuccinate, diamyl sulfosuccinate sodium, dicapryl sulfosuccinate sodium, diheptyl sulfosuccinate sodium, dihexyl sulfosuccinate sodium, diisobutyl sulfosuccinate sodium, ditridecyl sulfosuccinate sodium, sodium dodecylbenzenesulfonate, or a mixture thereof.

In another embodiment, the one or more intragranular excipients comprise a diluent comprising lactose monohydrate and microcrystalline cellulose; a disintegrant comprising sodium starch glycolate; a polymeric binder comprising polyvinylpyrrolidone; and a surfactant comprising sodium lauryl sulfate.

In one embodiment, the extragranular excipients are independently selected from the group consisting of a disintegrant, a glidant, a lubricant, and mixtures thereof. In certain embodiments, the extragranular excipients comprise a disintegrant, a glidant, and a lubricant.

In one embodiment, the extragranular excipients comprise: a disintegrant selected from the group consisting of low substituted hydroxypropyl cellulose, carboxymethylcellulose sodium, croscarmellose sodium, crospovidone, and sodium starch glycolate and mixtures thereof; a glidant selected from the group consisting of talc, calcium phosphate, calcium silicate, magnesium silicate, magnesium trisilicate, silicon dioxide, colloidal silicon dioxide, magnesium aluminosilicate, and mixtures thereof; and a lubricant selected from the group consisting of lauric acid, myristic acid, palmitic acid, stearic acid or pharmaceutically acceptable salts or esters thereof, such as magnesium stearate, calcium stearate, sodium stearyl fumarate, or zinc stearate or a mixture thereof.

In another embodiment, the one or more extragranular excipients comprise a disintegrant comprising sodium starch glycolate, a glidant comprising colloidal silicon dioxide, and a lubricant comprising magnesium stearate.

In another embodiment, the one or more intragranular components comprise a diluent comprising lactose monohydrate and microcrystalline cellulose; a diluent comprising sodium starch glycolate; a polymeric binder comprising polyvinylpyrrolidone; and a surfactant comprising sodium lauryl sulfate; and the one or more extragranular components comprise a disintegrant comprising sodium starch glycolate, a glidant comprising colloidal silicon dioxide, and a lubricant comprising magnesium stearate.

The pharmaceutical composition may have a total weight in the range of 250 mg to 1000 mg, such as from 400 mg to 1000 mg, such as 800 mg.

The pharmaceutical composition may be prepared according to the process comprising preparing a granulate comprising pretomanid and one or more intragranular excipients, and combining the granulate with one or more extragranular excipients to provide a blend. Embodiments of intragranular and extragranular excipients are as described above. The pharmaceutical composition may also be prepared according to the process comprising preparing a blend comprising a pharmaceutically effective amount of pretomanid and one or more pharmaceutically acceptable excipients.

A portion of such blends may be filled into a capsule shell or compressed to provide a solid dosage (e.g., tablets or caplets). The portion of the blend filled into a capsule or compressed contains a pharmaceutically effective amount of pretomanid. For example, a pharmaceutically effective amount of pretomanid includes an amount equivalent to 50 mg to 300 mg pretomanid, such as 100 mg or 200 mg pretomanid.

The granulate may be prepared, for example, by mixing a combination of pretomanid and the intragranular excipients, and subsequently granulating the mixture by wet or dry methods (including melt granulation) familiar to those skilled in the art to provide the granulate.

In one embodiment, the granulate is prepared by a dry granulation method in which pretomanid and one or more of the intragranular excipients (e.g., a binder, a diluent, and/or disintegrant) are dry mixed to form a dry mixture, and the mixture is subject to dry granulation by, for example, compaction and/or slugging. The resulting product of the dry granulation may be optionally milled to a desired particle size and/or sieved to assist with subsequent handling and/or processes as needed. Where a dry granulating process is utilized, the binder may comprise a dry binder such as pre-gelatinized starch, anhydrous lactose, dibasic calcium phosphate, or a mixture thereof.

In one embodiment, the granulate is prepared by a melt granulation method in which pretomanid and one or more of the intragranular excipients (e.g., a binder, a diluent, and/or disintegrant) are dry mixed to form a dry mixture, and the mixture is subject melt extrusion (e.g., in a single-or twin-screw extruder at a temperature suitable to melt the melt binder; for example 70-130° C.). The resulting product of the melt granulation may be optionally milled to a desired particle size and/or sieved to assist with subsequent handling and/or processes as needed. Where a melt granulating process is utilized, the binder may comprise a melt binder such as a polyethylene glycols (PEGs), poloxamer, fatty acid, fatty alcohol, wax, hydrogenated vegetable oil, and mixtures thereof.

In one embodiment, the granulate is prepared by a wet granulation method in which pretomanid and one or more of the intragranular excipients (e.g., a diluent and/or disintegrant) are dry mixed to form a dry mixture, a binder solution including a binder, a surfactant, and/or a solvent is added to the dry mixture to form a mixture, and the mixture is then subject to wet granulation. The wet granulation process may be performed using a high shear rapid mixer granulator. The resulting granules may be optionally dried and/or sieved to assist with subsequent handling and/or processes as needed.

According to one embodiment, the pharmaceutical composition may be prepared by a process which comprises: dry mixing a mixture comprising pretomanid and one or more intragranular excipients; adding a binder solution to the mixture; kneading the mixture to form wet granules; wet milling the kneaded mixture to provide granules; drying the granules; blending the granules with one or more extragranular excipients to provide a blend; and compressing the blend into tablets.

The dry mixing may be performed for 5 minutes to 15 minutes, such as for 10 minutes to achieve an appropriate blend uniformity. In some embodiments, the dry mixing may be performed with an impeller speed of 100 rpm. The binder solution may comprise water in the range of 30 to 60 wt. % of the dry mixture. In some embodiments, the binder addition time is about 2 to 3 minutes. The kneading step may be performed for 2 to 10 minutes, from 5 to 7 minutes, or for 3 to 6 minutes. In some embodiments, the granulation process is performed with an impeller speed of from 50 to 200 rpm, such as 100 rpm. In some embodiments, the granules may be dried for from 45 to 75 minutes.

In some embodiments, the blending step (blending the granules with one or more extragranular excipients to provide a blend) comprises a pre-lubrication step wherein the granules are blended with one or more extragranular excipients, such as a disintegrant and a glidant, to form a pre-lubrication blend, and a lubrication step wherein the pre-lubrication blend is blended with a lubricant to form a lubricated blend. The pre-lubrication step may be performed for from 5 to 15 minutes, such as for 5 minutes. The lubrication step may be performed for from 2 to 6 minutes, such as for 4 minutes.

The compressed tablets formed according the present disclosure may have a tablet hardness in the range of 4 to 17 Kp (Kilopond), such as from 4 to 17 Kp, from 4 to 10 Kp, from 10 to 16 Kp, from 4 to 10 Kp, from 7 to 13 Kp, from 7 to 8 Kp, or from 11 to 13 Kp. The tablets may have a friability of less than 0.8%, such as less than 0.2%.

Generally, the preceding dosage forms may be optionally coated (film-coated or non-film-coated). The film formers used for the coating process may, for example, be cellulose derivatives such as methyl cellulose (MC), ethyl cellulose (EC), hydroxyethyl cellulose (HEC), methacrylic acid/acrylate copolymers, HPMC, vinyl polymers or natural film formers, such as shellac. Examples of commercially available film formers include, but are not limited to, Opadry® (HPMC), Opadry® II (poly (vinyl alcohol)), and Surelease® (Ethylcellulose Dispersion Type B NF) Film Coating Systems (each available from Colorcon, Inc., North Wales, Pennsylvania), and mixtures thereof. In some embodiments, the dosage forms are uncoated.

The granulate of the pharmaceutical compositions of the present disclosure may have a particle size distribution such that no more than 30 wt %, such as no more than 25 wt %, such as no more than 20 wt %, such as no more than 15 wt %, such as no more than 10 wt. % of the granulate is retained on an American Standard Test Sieve Series (ASTM) #60 sieve screen, e.g., having a particle size greater than 250 μm. The granulate of the pharmaceutical compositions of the present disclosure may have a particle size distribution such that from 5 to 30 wt. % of the granulate is retained on the #60 sieve, such as from 9 wt. % to 25 wt. % or from 10 wt. % to 25 wt. %. The granulate of the pharmaceutical compositions of the present disclosure may have a particle size distribution wherein about 5 wt. %, about 10 wt. %, about 15 wt. %, about 20 wt. %, about 25 wt. %, or about 30 wt. % of the granulate is retained on the #60 sieve. The granulate of the pharmaceutical compositions of the present disclosure may have a particle size distribution wherein a range from about 5 wt. % to about 30 wt. %, about 10 wt. % to about 30 wt. %, about 10 wt. % to about 25 wt. %, or about 5 wt. % to about 25 wt. % of the granulate is retained on the #60 sieve.

The granulate of the pharmaceutical composition may also have a particle size distribution such that at least 80 wt. % of the granulate is retained on an ASTM #200 sieve, e.g., having a particle size greater than 75 μm.

The lubricated blend of the pharmaceutical compositions of the present disclosure may have a particle size distribution such that no more than 30 wt %, such as no more than 25 wt %, such as no more than 20 wt %, such as no more than 15 wt %, such as no more than 10 wt. % of the lubricated blend is retained on an American Standard Test Sieve Series (ASTM) #60 sieve screen, e.g., having a particle size greater than 250 μm. The lubricated blend of the pharmaceutical compositions of the present disclosure may have a particle size distribution such that from 5 to 30 wt. % of the lubricated blend is retained on the #60 sieve, such as from 9 wt. % to 25 wt. % or from 10 wt. % to 25 wt. %.

The lubricated blend of the pharmaceutical composition may also have a particle size distribution such that at least 80 wt. % of the lubricated blend is retained on an ASTM #200 sieve, e.g., having a particle size greater than 75 μm.

The granulate of the pharmaceutical compositions of the present disclosure may have a bulk density within the range of 0.3 g/mL to 0.8 g/mL, such as 0.45 g/mL to 0.58 g/mL, or 0.47 to 0.58 g/ml, or 0.47 to 0.53 g/mL, or 0.47 to 0.526 g/mL, or 0.50 to 0.58 g/mL, or 0.50 to 0.55 g/mL, or 0.50 to 0.53 g/mL, or 0.50 to 0.526 g/mL.

The lubricated blend of the pharmaceutical compositions of the present disclosure may have a bulk density within the range of 0.3 g/mL to 0.8 g/mL, such as 0.45 g/mL to 0.58 g/mL, or 0.47 to 0.58 g/ml, or 0.47 to 0.53 g/mL, or 0.47 to 0.526 g/mL, or 0.50 to 0.58 g/mL, or 0.53 to 0.58 g/mL.

Bulk densities can be determined according to methods familiar to those skilled in the art. For example, a quantity of powder is passed through a sieve with apertures greater than or equal to 1.0 mm, if necessary, to break up agglomerates. Then, approximately 100 g of the test sample (m; weighed with 0.1% accuracy) is gently passed into a dry graduated cylinder (e.g., of 250 mL, readable to 2 mL), without compacting. The powder is carefully leveled, without compacting, and the unsettled apparent volume (V0) is read to the nearest graduated unit. The bulk density in (g/ml) is calculated using the formula m/V0.

In certain embodiments, the pharmaceutical compositions comprising pretomanid prepared according to the present disclosure were found to exhibit a dissolution profiles suitable for an orally administered immediate release composition. An “immediate release” composition, as described herein, refers to a composition that is formulated to rapidly release the active drug (API) after oral administration. Rate of API release can be measured in a USP Apparatus 2 (Paddle Apparatus) at 37+/−2° C., according to the methods of USP42-NF37 chapter <711> on dissolution, which is incorporated herein by reference. For example, a suitable dissolution medium used with USP Apparatus 2 can be 0.5% hexadecyltrimethylammonium bromide (HDTMA) in 0.1N HCl. In certain embodiments, “immediate release” means that the composition releases greater than about 40 wt. % or greater than 50 wt. %; or greater than about 55 wt. %; or greater than about 60 wt. % of the API within 30 minutes within the preceding method and dissolution medium. In certain embodiments, “immediate release” means that the composition releases greater than 40 wt. % or greater than about 50 wt. %; or greater than about 55 wt. %; or greater than about 60 wt. % of the API within 20 minutes within the preceding method and dissolution medium

In some embodiments of the compositions herein, at least 40 wt. % or at least 50 wt. % or at least 60 wt. % or at least 65 wt. % or at least 70 wt. % or at least 75 wt. % of the pretomanid in the pharmaceutical composition dissolves within 20 minutes in a dissolution medium comprising 0.5% hexadecyltrimethylammonium bromide (HDTMA) in 0.1N HCl.

In other embodiments, at least 40 wt. % or at least 50 wt. % or at least 60 wt. % or at least 65 wt. % of the pretomanid in the pharmaceutical composition dissolves within 10 minutes in 0.5% HDTMA in 0.1N HCl, such as at least 70 wt. %, at least 75 wt. %, or at least 80 wt. %.

In other embodiments, at least 75 wt. % or at least 80 wt. % or at least 85 wt. % or at least 90 wt. % or at least 95 wt. % of the pretomanid in the pharmaceutical composition dissolves within 40 minutes in 0.5% HDTMA in 0.1N HCl.

The pharmaceutical compositions of the present disclosure may have a disintegration time of less than 10 minutes as measured at 37±2° C. according to the methods of USP42-NF37 chapter <701> on disintegration. For example, in some embodiments, the pharmaceutical composition has a disintegration time of less than or equal to 5 minutes, or less than or equal to 3 minutes.

In other embodiments, the composition have pretomanid dissolution and composition disintegration times according to any one of the following embodiments listed in Table 1, as measured by USP chapters <711> and <701>, respectively:

TABLE 1

| (#) | Dissolution | Disintegration |
|---|---|---|
| (1) | >40 wt % in 10 minutes | <5 minutes |
| | and >60 wt % in 20 minutes | |
| (2) | >40 wt % in 10 minutes | <5 minutes |
| | and >60 wt % in 20 minutes | |
| | and >80 wt % in 40 minutes | |
| (3) | >60 wt % in 10 minutes | <5 minutes |
| | and >80 wt % in 20 minutes | |
| (4) | >60 wt % in 10 minutes | <5 minutes |
| | and >80 wt % in 20 minutes | |
| | and >90 wt % in 40 minutes | |
| (5) | >65 wt % in 10 minutes | <5 minutes |
| | and >85 wt % in 20 minutes | |
| | and >95 wt % in 40 minutes | |
| (6) | >40 wt % in 10 minutes | <3 minutes |
| | and >60 wt % in 20 minutes | |
| (7) | >40 wt % in 10 minutes | <3 minutes |
| | and >60 wt % in 20 minutes | |
| | and >80 wt % in 40 minutes | |
| (8) | >60 wt % in 10 minutes | <3 minutes |
| | and >80 wt % in 20 minutes | |
| (9) | >60 wt % in 10 minutes | <3 minutes |
| | and >80 wt % in 20 minutes | |
| | and >90 wt % in 40 minutes | |
| (10) | >65 wt % in 10 minutes | <3 minutes |
| | and >85 wt % in 20 minutes | |
| | and >95 wt % in 40 minutes | |

The pharmaceutical composition of the present disclosure can be useful for the treatment of tuberculosis. A “therapeutically effective amount” is an amount effective for treating a recited disease or condition, such as, tuberculosis. The term “treating” or “treatment”, with regard to a subject, refers to improving at least one symptom of the subject's disorder. Treating can be curing, improving, or at least partially ameliorating the disorder.

In one embodiment, a method for treating tuberculosis comprises administering the pharmaceutical composition described herein to a patient in need of such treatment. In particular, the pharmaceutical composition may be administered to a patient orally.

In one embodiment, a method for treating tuberculosis further comprises co-administering to a patient one or more separate dosage forms, each comprising therapeutically effective amounts of active pharmaceutical ingredients (APIs) effective for the treatment of tuberculosis. Such co-administration may be simultaneous or sequential. Suitable separate dosage forms may be selected from commercially available dosage forms containing APIs selected from the group consisting of bedaquiline (e.g. bedaquiline fumarate as Sirturo® tablets, 100 mg base, available from Janssen Theraps.), moxifloxacin (e.g., moxifloxacin hydrochloride as Avelox® oral tablets, 400 mg base, available from Bayer Healthcare Pharma. Inc.), linezolid (e.g., as Zyvox® oral tablets, 600 mg, available from Pharmacia and Upjohn Company LLC), pyrazinamide (e.g., pyrazinamide oral tablets, 500 mg, available from Akorn Inc.), or pharmaceutically acceptable salts of the preceding.

In one particular embodiment, bedaquiline fumarate tablets and linezolid oral tablets, are co-administered with the pretomanid formulations herein. In another embodiment bedaquiline fumarate tablets, moxifloxacin hydrochloride oral tablets, and pyrazinamide oral tablets are co-administered with the pretomanid formulations herein.

Therapeutically effective amounts of the APIs here can include daily dosages in the ranges of 50 mg-2000 mg pyrazinamide; 10 mg-800 mg moxifloxacin; 10 mg-400 mg pretomanid (e.g., 100 mg or 200 mg); 100 mg-2000 mg linezolid (e.g., 600 mg or 1200 mg); and 10 mg-400 mg bedaquiline (e.g., 200 mg or 400 mg); amounts of moxifloxacin and bedaquiline may be calculated based on the free base, but may be present as equivalent amount of bedaquiline fumarate and/or moxifloxacin hydrochloride.

The method of treatment of the present disclosure may further comprise administering the pharmaceutical composition described herein for a duration or frequency sufficient to treat tuberculosis. For example, a therapeutically effective amount of pretomanid, such as 200 mg of pretomanid, may be orally administered once daily for 26 weeks. The method of treatment may additionally include co-administration of bedaquiline dosage forms and linezolid dosage forms. For example, according to one non-limiting embodiment, the method of treatment further includes orally administering a dosage form comprising 400 mg (base) of bedaquiline once daily for two weeks followed by a dosage form comprising 200 mg (base) of bedaquiline three times per week (with at least 48 hours between doses) for a total of 26 weeks and orally administering a dosage form comprising 1200 mg of linezolid daily for up to 26 weeks. According to certain non-limiting embodiments, missed doses of the pretomanid-bedaquiline-linezolid regimen can be made up at the end of treatment.

In some embodiments, the pharmaceutical composition of the present disclosure can be used for the treatment of drug-susceptible tuberculosis (DS-TB). DS-TB refers to TB which is not resistant to any of the TB drugs. In some embodiments, the pharmaceutical composition of the present disclosure can be used for the treatment of multi-drug resistant tuberculosis (MDR-TB). MDR-TB refers to TB caused by bacteria resistant to two of the most important first-line TB medicines, isoniazid (INH) and rifampin (RIF). In some embodiments, the pharmaceutical composition of the present disclosure can be used for the treatment of extensively-drug resistant tuberculosis (XDR-TB). XDR-TB refers to a rare type of MDR-TB that is resistant to INH and RIF, and is additionally resistant to any fluoroquinolone, such as ciprofloxacin, levofloxacin, ofloxacin, or sparfloxacin, and at least one of three injectable second-line drugs, such as amikacin, kanamycin, or capreomycin.

The following examples are presented to demonstrate the general principles of the invention of this disclosure. The invention should not be considered as limited to the specific examples presented. All parts and percentages in the examples are percent weight, based on the total weight of the pharmaceutical composition, unless otherwise indicated.

### EXAMPLES

### Example 1 Pretomanid Composition

Table 2 shows an example composition (Example 1) of the present disclosure.

TABLE 2

| | | |
|---|---|---|
| | | Total batch |
| | | quantity |
| Ingredients | mg/unit | (kg) |

Dry intragranular materials

| | | |
|---|---|---|
| Pretomanid | 200.0 | 15.00 |
| Lactose monohydrate | 294.4 | 22.08 |
| Cellulose Microcrystalline | 235.2 | 17.64 |
| Sodium Starch Glycolate | 20.0 | 1.50 |

Binder solution materials

| | | |
|---|---|---|
| Povidone | 16.0 | 1.20 |
| Sodium Lauryl Sulfate | 4.0 | 0.30 |
| Purified Water¹ | q.s. | 22.50 |
| | (30-60% of | |
| | dry mix) | |

Extragranular materials

| | | |
|---|---|---|
| Sodium Starch Glycolate | 20.0 | 1.50 |
| Colloidal Silicon Dioxide | 2.4 | 0.18 |
| Magnesium Stearate | 8.0 | 0.60 |
| Total Weight | 800 | 60 |

¹Purified water will not appear in the final product except in traces

### Manufacturing Procedure

1. Dry intragranular materials (pretomanid, lacotose monohydrate, cellulose microcrystalline, and sodium starch glyocolate) were passed through #16 ASTM and collected in polybag.
2. Required quantity of water was transferred in beaker. Povidone (K-30) was dispersed in purified water and stirred until clear solution was formed. Sodium Lauryl Sulfate was added to povidone solution and stirred until a clear binder solution was formed. Care was taken to avoid entrapment of air.
3. Sifted materials of step 1 were transferred to rapid mixer granulator.
4. Blend of step 3 was dry mixed for 5 minutes.
5. Binder solution of step 2 was transferred into blend of step 4 with varying impeller speed.
6. Wet mass of step 5 was kneaded for varying time at varying speed of impeller as per the design. The granules were unloaded into polybag.
7. The wet mass of step 6 was passed through Quadro co-mill using 375Q at 1200 rpm.
8. Granules of step 7 were dried using fluidized bed dryer at an inlet air temperature of 50° C. until loss-on-drying (LOD) of not more than 1.5% w/w is achieved.
9. Dried granules of step 8 were passed through Quadro co-mill using 50G screen at 1200 rpm.
10. Sodium starch glycolate and colloidal silicon dioxide were passed through #20 ASTM sieve and blended with milled granules of step 9 in blender for 10 min at 20 rpm.
11. Magnesium stearate were passed through #60 ASTM sieve (250 μm) was transferred to granules of step No.10. Granules were lubricated for 4 min at 20 rpm in a blender.
12. The lubricated blend of step 11 was compressed to form tablets.

### Example 2 Wet Granulation Process Effect on Dissolution and Disintegration of Pretomanid Tablets

TABLE 3

| | | | | % | | | Disin- |
|---|---|---|---|---|---|---|---|
| | | | | Retained | | | tegra- |
| | Water | kneading | Impeller | on # 60 | Bulk | % | tion |
| Run | uptake | time | speed | (ASTM) | Density | Dissolution | Time |
| No. | (%) | (min.) | (rpm) | (%) | (g/mL) | (10 min.) | (min) |
| 1 | 30 | 10 | 200 | 8.9 | 0.526 | 78 | 1.75 |
| 2 | 60 | 2 | 50 | 29.3 | 0.500 | 72 | 2.92 |
| 3 | 30 | 2 | 200 | 11.7 | 0.517 | 82 | 1.75 |
| 4 | 30 | 2 | 50 | 16.2 | 0.476 | 78 | 1.50 |
| 5 | 60 | 2 | 200 | 66.3 | 0.517 | 38 | 4.25 |
| 6 | 60 | 10 | 200 | 80.7 | 0.588 | 4 | 18.33 |
| 7 | 60 | 10 | 50 | 49.7 | 0.526 | 48 | 4.25 |
| 8 | 30 | 10 | 50 | 11.7 | 0.526 | 83 | 2.00 |

In runs #1-8 shown in Table 3 using the formulation and process of Table 2 (supra), factorial designed array of variations on water uptake (30-60%), kneading time (2-10 minutes), and impeller speed (50-200 rpm) were examined for the effect on the resultant granulate and tablets formed therefrom (Table 2).

It was observed that as the % water uptake was increased, there was a corresponding increase in the % granulate retained on #60 ASTM, which was further enhanced by increasing the kneading time. Similarly, when water uptake was increased with increased in impeller speed there was a significant increase in the % granulate retained on #60 ASTM. The effect of impeller speed was comparatively more than the effect of kneading time. Increase in % water uptake, kneading time and impeller speed also showed corresponding increase in bulk density of the granulate. At a fixed impeller speed, as both the remaining two factors (% water uptake and kneading time) increased, there was significant decrease in dissolution rate. Similarly, at a fixed kneading time, the dissolution rate was decreased with increase an increase in impeller speed, but this was greatly enhanced by an increasing water uptake. With an increase in kneading time and impeller speed at a fixed water uptake level, there was a decrease in dissolution rate.

Notably, both improved disintegration (less than 3 minutes) and dissolution (greater than 70% at 10 minutes (in 0.5% HDTMA in 0.1N HCl, USP-II (paddle), 75 rpm, 1000 mL) were observed when bulk density of the granulate (prior to lubrication) was less than about 0.53 g/mL and retention on #60 (ASTM) sieve was less than about 30%.

### Example 3 Bulk Pretomanid Tablet Formulation and Process

One batch of 60.0 kg common blend, having the composition of Table 2, was manufactured according to the following:

### Sifting

Pretomanid, lactose monohydrate (spray dried grade; SuperTab® 11SD, DFE Pharma, Goch, Germany), microcrystalline cellulose (Avicel® PH-102), sodium starch glycolate (Type-A) intra granular were co-sifted through Quadro sifter fitted with 75R screen at a speed of 700 RPM and the sifted materials were collected in containers lined with double polybag.

Colloidal silicon dioxide (Aerosil 200) and sodium starch glycolate (Type-A) extra granular were co-sifted through Quadro sifter fitted with 55R screen at a speed of 700 RPM and the sifted materials were collected in containers lined with double polybag.

Magnesium stearate was sifted through Quadro sifter fitted with 18R screen at a speed of 700 RPM and the sifted material was collected in containers lined with double polybag.

The raw materials were free of lumps and foreign matter before and after sifting.

### Dry Mixing

Sifted pretomanid, lactose monohydrate (SuperTab® 11SD), microcrystalline cellulose (Avicel® PH-102), sodium starch glycolate (Type-A) intragranular were loaded into High Shear Mixer Granulator (Rapid Mixer Granulator-RMG) and mixed at an impeller speed of 60 RPM and chopper off for between 5 and 15 minutes until suitable blend uniformity.

### Wet Granulation

Binder solution was prepared by adding povidone (PVP K-30) to purified water under stirring using a pneumatic stirrer until a clear solution was formed. Sodium lauryl sulfate (Texapon K12 P) was added to the above solution and stirred until a clear solution was formed. The binder solution was added to the dry mixed material in the rapid mixer granulator over three minutes (poured, not sprayed because of the volumes involved) followed by kneading for six minutes at an impeller speed of 60 RPM and chopper off. Granules of good texture were obtained at the end of the granulation process which were assessed when a portion of wet mass was taken, compacted in the hand and could be easily fractionated with the fingers.

### Wet Milling

Using an impeller speed of 50 RPM the wet granules of the above step were passed through 250Q screen fitted to an in-line Quadro Comil at 720 RPM and milled granules were directly transferred to the fluid bed equipment through the vacuum conveying system for drying.

### Drying

Wet granules were dried in fluid bed equipment. The parameters recorded during the process of drying are presented in Tables 4 and 5.

TABLE 4
Process parameters for drying

| | | Initial | Observed |
|---|---|---|---|
| | | Batch | Range |
| | | production | (During |
| Parameter | Target | record range | execution) |
| A. Fluidization air (cfm) | 600 | 300-1700 | 491-1229 |
| B. Inlet air temperature (° C.) | 50 | 40-60 | 49-55 |
| C. Product temperature (° C.) | 30 | 25-48 | 18-38 |
| D. Filter shaking interval (sec) | 10 | 6-16 | 6-16 |
| E. Filter shaking pause interval (sec) | 120 | 60-240 | 60-240 |

TABLE 5
Drying parameters

| | | Inlet air | Outlet air | Product | | |
|---|---|---|---|---|---|---|
| Sam- | | temper- | temper- | temper- | | LOD |
| ple | Time | ature | ature | ature | Fluidization | % |
| No. | (minutes) | (° C.) | (° C.) | (° C.) | Air (cfm) | w/w |
| 1 | 0 | NA | NA | NA | NA | NA |
| 2 | 15 | 51 | 21 | 21 | 739 | 21.53 |
| 3 | 30 | 55 | 23 | 18 | 491 | 22.47 |
| 4 | 45 | 50 | 22 | 23 | 1229 | 16.19 |
| 5 | 60 | 50 | 23 | 24 | 901 | 9.32 |
| 6 | 75 | 49 | 24 | 26 | 755 | 3.90 |
| 7 | 90 | 49 | 34 | 38 | 736 | 1.56 |

After drying, samples were collected and loss on drying (LOD) was determined. The loss on drying of the samples collected was less than 2.0% w/w, ranging from 1.18 to 1.36%.

### Dry Milling

Dried granules of the above step were passed through 50G screen fitted to a Quadro Comil at 700 RPM and milled granules were collected into containers lined with double polybag. After the milling, one pooled sample was collected, analyzed for particle size distribution and the details are presented in Table 6.

TABLE 6
Particle size distribution of the dried granules
Particle size distribution

| Sieve No. # | Cumulative % weight retained |
|---|---|
| 20 | 0.58 |
| 40 | 7.71 |
| 60 | 14.83 |
| 80 | 24.32 |
| 100 | 37.83 |
| 200 | 84.89 |
| Pan | 100.25 |

The dried granules passed easily through the Quadro Comil and there were no retains in the screen fitted to the Quadro Comil. The cumulative % weight retained on #60 (250 μm) and #200 (75 μm) screens was 14.8% and 84.9% respectively.

Milled granules were blended with the sifted extragranular material (colloidal silicon dioxide (Aerosil® 200) and sodium starch glycolate (Type-A)) for a suitable time between 3 and 7 minutes in a bin blender at 10 RPM. Sifted magnesium stearate was added to the pre-lubricated granules in the bin blender and lubricated for a suitable time between 2 and 6 minutes. After 4 minutes of lubrication, a sample for the determination of bulk density, particle size distribution, loss on drying (LOD) and assay was withdrawn and the observations are presented in Table 7.

TABLE 7
Apparent and Tapped Bulk density of Lubricated Blend
Density (g/mol)

| Parameters | Observations |
|---|---|
| Bulk density | 0.569 |
| Tapped density | 0.687 |
| Compressibility index | 17.176 |

Particle Size Distribution

| Sieve size # | Cumulative weight % retained |
|---|---|
| 20 | 0.49 |
| 40 | 7.21 |
| 60 | 14.17 |
| 80 | 25.37 |
| 100 | 38.18 |
| 200 | 85.53 |
| Pan | 100.12 |
| Loss on drying % | 1.37 |
| Assay (%) | 100.5 |

The lubricated blend was divided into two parts, Part A: 20.00 kg of blend for pretomanid tablets 100 mg and Part B: 40.00 kg of blend for pretomanid tablets 200 mg.

### 100 mg Tablets

The lubricated blend (Part A) was compressed into pretomanid tablets 100 mg, using 14.5×5.6 mm Modified capsule shaped, deep concave punches, embossed with ‘T100’ on one side and plain on the other side, with corresponding dies.

Pretomanid tablets 100 mg were compressed in a 30 station compression machine at two different compression speeds, 300 tablets per minute (TPM) & 1500 TPM. Samples were collected at each compression speed and tested for description, weight variation, disintegration time, thickness, hardness and friability. A pooled sample was collected from each compression speed and ten selected and tested for uniformity of dosage units. The results are presented in Table 8.

TABLE 8
Tablet characteristics at different compression speeds

| Com- | Weight | | | Disin- | | |
|---|---|---|---|---|---|---|
| pression | of 20 | Hard- | Thick- | tegration | | Unifor- |
| speed | tablets | ness | ness | time | % | mity |
| (TPM) | (g) | (Kp) | (mm) | (min.) | Friability | (% RSD) |
| 300 | 8.037 | 7 | 5.28-5.47 | 2.5 | 0.07 | 99.4 (0.48) |
| 1500 | 8.040 | 7-8 | 5.28-5.66 | 2.5 | 0.06 | 98.2 (0.92) |

Change of compression speed within the range of 300 TPM to 1500 TPM, did not have any significant effect on the hardness, thickness, weight uniformity, disintegration, friability and content uniformity of compressed tablets. The acceptance values are less than 5.0 at both the speeds.

Pretomanid tablets 100 mg were compressed on a 30 station compression machine at the target speed of 900 TPM. In order to evaluate the effect of hardness on tablet characteristics, tablets were compressed at varying hardnesses.

Samples were collected and tested for description, weight variation, thickness, disintegration time and friability. A pooled sample was collected from each hardness range and tested for dissolution. The results are presented below in Tables 9 and 10. The dissolution medium was 0.5% HDTMA in 0.1N HCl, USP-II apparatus, paddle stirrer at 75 rpm, 1000 mL volume.

TABLE 9
Tablet characteristics at different hardness

| | | Weight | | | |
|---|---|---|---|---|---|
| | Compression | of 20 | | Disintegra- | |
| Hardness | speed | tablets | Thickness | tion time | % |
| (Kp) | (TPM) | (g) | (mm) | (min.) | Friability |
| 5-7 | 900 | 8.026 | 5.28-5.47 | 1.25 | 0.15 |
| 7-8 | 900 | 8.038 | 5.28-5.47 | 2.5 | 0.09 |
| 10-11 | 900 | 8.017 | 5.03-5.10 | 6.0 | 0.09 |

TABLE 10
Effect of Hardness on Dissolution

| | % wt. Drug Release | | | | | |
|---|---|---|---|---|---|---|
| | 5-7 Kp Hardness | | 7-8 Kp Hardness | | 10-11 Kp Hardness | |
| Time | | % | Range | % | Range | % |
| (min.) | Range (mean) | RSD | (mean) | RSD | (mean) | RSD |
| 10 | 66-74 (68) | 4.6 | 67-69 (69) | 1.2 | 36-43 (40) | 6.3 |
| 20 | 82-88 (84) | 2.7 | 81-87 (85) | 2.6 | 66-69 (67) | 1.9 |
| 40 | 92-99 (95) | 3.1 | 93-98 (96) | 0.8 | 84-87 (86) | 1.4 |
| 60 | 99-99 (99) | 0.0 | 98-100 (99) | 0.8 | 92-94 (93) | 0.8 |
| 120 | 100-101 (100) | 0.5 | 99-101 (101) | 0.8 | 97-100 (99) | 1.0 |

### 200 mg Tablets

The lubricated blend (Part B) was compressed in to pretomanid tablets 200 mg, using 17.9×8.9 mm Oval shaped concave punches embossed ‘T200’ on one side and plain on the other side with corresponding dies.

Pretomanid tablets 200 mg were compressed on a 30 station compression machine at two different compression speeds, 300 TPM & 1500 TPM. Samples were collected at each compression speed and tested for description, weight variation, hardness, thickness, disintegration time (DT) and friability. A pooled sample was collected from each compression speed and ten selected and tested for uniformity of dosage units. The results are presented in Table 11.

TABLE 11
Tablet characteristics at different compression speeds

| Com- | Weight | | | | | Unifor- |
|---|---|---|---|---|---|---|
| pression | of 20 | Hard- | Thick- | Dis- | | mity |
| speed | tablets | ness | ness | integration | % | (% |
| (TPM) | (g) | (Kp) | (mm) | time | Friability | RSD) |
| 300 | 16.04 | 10-14 | 5.95-6.14 | 2 min. 50 sec. | 0.09 | 99.7 (0.48) |
| 1500 | 16.09 | 12-13 | 6.02-6.10 | 2 min. 35 sec. | 0.07 | 99.9 (0.73) |

Change of compression speed within the range of 300 TPM to 1500 TPM, did not have any significant effect on the hardness, thickness, weight uniformity, disintegration, friability and content uniformity of compressed tablets. The acceptance values are less than 5.0 at both the speeds.

Pretomanid tablets 200 mg were compressed on a 30 station compression machine at the target speed of 900 TPM. In order to evaluate the effect of hardness on tablet characteristics, tablets were compressed at varying hardnesses. Samples were collected and tested for description, weight variation, thickness, disintegration time and friability. A pooled sample was collected from each hardness range and tested for dissolution. The results are presented below in Tables 12 and 13. The dissolution medium was 0.5% HDTMA in 0.1N HCl, USP-II (paddle), 75 rpm, 1000 mL.

TABLE 12
Tablet characteristics at different hardness

| | | Weight | | | |
|---|---|---|---|---|---|
| | Compression | of 20 | | Disintegra- | |
| Hardness | speed | tablets | Thickness | tion time | % |
| (Kp) | (TPM) | (g) | (mm) | (min.) | Friability |
| 12-14 | 900 | 16.05 | 6.05-6.20 | 2.5 | 0.09 |
| 14-16 | 900 | 16.03 | 5.92-5.98 | 6.0 | 0.07 |
| 11-13 | 900 | 16.08 | 6.05-6.08 | 3.0 | 0.07 |

TABLE 13
Effect of Hardness on Dissolution

| | % wt. Drug Release | | | | | | | |
|---|---|---|---|---|---|---|---|---|
| | 12-14 Kp | | 14-16 Kp | | 11-13 | | 7-8 Kp | |
| | Hardness | | Hardness | | Kp Hardness | | Hardness | |
| Time | Range | % | Range | % | Range | % | Range | % |
| (min.) | (mean) | RSD | (mean) | RSD | (mean) | RSD | (mean) | RSD |
| 10 | 48-53 | 3.1 | 39-44 | 5.0 | 48-51 | 2.6 | 60-64 (62) | 2.7 |
| | (51) | | (42) | | (50) | | | |
| 20 | 70-74 | 2.3 | 62-88 | 3.1 | 64-71 | 3.5 | 77-81 (79) | 1.7 |
| | (72) | | (66) | | (67) | | | |
| 40 | 85-91 | 2.7 | 81-84 | 1.3 | 82-87 | 1.0 | 90-93 (92) | 1.4 |
| | (87) | | (83) | | (84) | | | |
| 60 | 91-97 | 2.4 | 89-92 | 1.3 | 91-94 | 1.2 | 95-97 (96) | 1.1 |
| | (95) | | (91) | | (93) | | | |
| 120 | 95-101 | 2.2 | 96-99 | 1.2 | 97-101 | 1.5 | 98-101 | 1.3 |
| | (99) | | (98) | | (100) | | (100) | |

### Example 4 Pharmacokinetic Analyses

Tables 14 and 15 show the rate and extent of absorption as measured by Tₘₐₓ, Cₘₐₓ, AUCₜ, and AUC_{∞} of a single oral dose of the pharmaceutical composition of the present invention (200 mg pretomanid in tablet form) in healthy adult male and female subjects when administered approximately 30 minutes after a high-calorie, high-fat meal (fed state) and when administered after a minimum 10-hour fast (fasted state). Fed state subjects were provided with a standard high-calorie, high-fat meal per USFDA guidance which is “two eggs fried in butter, two strips of bacon, two slices of toast with butter, four ounces of hash brown potatoes and eight ounces of whole milk.” Substitutions were allowed as long as the meal provided a similar amount of calories from protein, carbohydrate, and fat and has a comparable meal volume and viscosity. Fasting subjects were required to fast for a minimum of 10 hours overnight prior to each dose and for at least 4 hours thereafter, and excluded from fluids from one hour before until one hour after dosing.

16 subjects were divided into two treatment groups: Group 1 received one dose of 200 mg pretomanid in the fasted state and another dose of pretomanid in the fed state approximately 8 days later; and Group 2 were dosed in parallel with Group 1, but in the opposite order, i.e., first in the fed state and then in the fasted state. Both groups were housed and treated in parallel. Approximately 4 men and 4 women were in each group. Subjects received their dosages with 240 mL water and were required to drink all 240 mL of water.

Each pharmacokinetic parameter shown in Tables 14 and 15 was determined separately using plasma concentrations of pretomanid following dosing in the fasted vs. fed states by applying a noncompartmental approach using PK software such as WinNonlin Professional. Blood samples (10 ml) were collected at the following times: predose, 0.5, 1, 2, 3, 4, 6, 8, 12, and 16 hours after dosing on Days 1 and 9; 24 and 36 hours after Day 1 and Day 9 dosing (i.e., during days 2 and 10), and daily on Days 3 through 5 and 11 through 13 at the approximate time dosing would have occurred. Plasma samples were analyzed for pretomanid using an LC/MS/MS (liquid chromatography-tandem mass spectrometry) method.

“Cₘₐₓ” refers to the maximum observed drug concentration. “Tₘₐₓ” refers to the observed time of the maximum drug concentration (obtained without interpolation). “AUCₜ” refers to area under the drug concentration-time curve calculated using linear trapezoidal summation from time zero to time t, wherein t is the time of the last measurable concentration (Ct). “AUC_{∞}” refers to the area under the drug concentration-time curve from time zero to infinity, wherein AUC_{∞}=AUCₜ+Ct/Kel. “Kel” refers to the apparent terminal elimination rate constant calculated by linear regression of the terminal linear portion of the log concentration vs. time curve. The arithmetic mean of each parameter and % CV (coefficient of variation) were determined for the fed and the fasted state, as shown in tables 14 and 15.

TABLE 14

Arithmetic Mean (% CV)
(N = 16)

| Pharmacokinetic | Fasted Healthy | Fed Healthy |
|---|---|---|
| Parameters | Volunteers | Volunteers |
| Cₘₐₓ (μg/mL) | 1.13 (18.9) | 1.97 (15.1) |
| AUCₜ (μg · hr/mL) | 28.1 (28.5) | 51.6 (19.6) |
| AUC_{∞} (μg · hr/mL) | 28.8 (28.7) | 53.0 (20.1) |

TABLE 15
Tₘₐₓ (hours) at 200 mg Single Dose
Arithmetic Mean (range), (N = 16)

| Fasted Healthy Volunteers | Fed Healthy Volunteers |
|---|---|
| 4.06 (2.00-6.00) | 5.07 (3.00-8.07) |

Whereas particular embodiments of this invention have been described above for purposes of illustration, it will be evident to those skilled in the art that numerous variations of the details of the present invention may be made without departing from the invention as defined in the appended claims.

1. An oral pharmaceutical composition comprising a granulate comprising a pharmaceutically effective amount of pretomanid or a pharmaceutically acceptable solvate thereof, wherein the granulate has a bulk density in a range of about 0.3 to 0.8 g/mL, wherein the granulate has a particle size distribution such that no more than about 30 wt. % of the granulate is retained on an ASTM #60 (250 μm) sieve, and wherein at least 40 wt. % of the pretomanid is dissolved within 20 minutes as measured in a USP-II Apparatus at 37±2° C. in 0.5% hexadecyltrimethylammonium bromide (HDTMA) in 0.1N HCl.

2. The pharmaceutical composition of claim 1, wherein the bulk density range is about 0.47 to about 0.53 g/mL.

3. The pharmaceutical composition of claim 1, wherein the particle size distribution is such that about 5 wt. % to about 30 wt. % of the granulate is retained on the ASTM #60 (250 μm) sieve.

4. The pharmaceutical composition of claim 1, wherein the particle size distribution is such that at least 80 wt. % of the granulate is retained on an ASTM #200 (75 μm) sieve.

5. The pharmaceutical composition of claim 1, wherein the composition has a disintegration time of less than 10 minutes (e.g., less than or equal to 5 minutes).

6. The pharmaceutical composition of claim 1, wherein at least 60 wt. % of the pretomanid is dissolved within 10 minutes in 0.5% HDTMA in 0.1N HCl, or at least 75 wt. % of the pretomanid is dissolved within 40 minutes in 0.5% HDTMA in 0.1N HCl.

7-8. (canceled)

9. The pharmaceutical composition of claim 1, wherein the pretomanid comprises from 10 wt. % to 30 wt. % of the pharmaceutical composition.

10. The pharmaceutical composition of claim 1, comprising the granulate and one or more pharmaceutically acceptable extragranular excipients, wherein the granulate comprises the pretomanid and one or more pharmaceutically acceptable intragranular excipients.

11. The pharmaceutical composition of claim 10, wherein each excipient is independently selected from the group consisting of a diluent, a disintegrant, a binder, a surfactant, a glidant, and a lubricant.

12. (canceled)

13. The pharmaceutical composition of claim 11, wherein each diluent is selected from the group consisting of a saccharide, a disaccharide, a sugar alcohol, a polysaccharide, and a polysaccharide derivative.

14. (canceled)

15. The pharmaceutical composition of claim 11, wherein the diluent comprises lactose monohydrate (e.g. spray-dried lactose monohydrate) and microcrystalline cellulose.

16. The pharmaceutical composition of claim 11, wherein the binder comprises a polymeric binder.

17. (canceled)

18. The pharmaceutical composition of claim 11, wherein the surfactant comprises sodium lauryl sulfate, ammonium lauryl sulfate, docusate sodium, ammonium dinonyl sulfosuccinate, diamyl sulfosuccinate sodium, dicapryl sulfosuccinate sodium, diheptyl sulfosuccinate sodium, dihexyl sulfosuccinate sodium, diisobutyl sulfosuccinate sodium, ditridecyl sulfosuccinate sodium, sodium dodecylbenzenesulfonate, or a mixture thereof.

19-20. (canceled)

21. The pharmaceutical composition of claim 11, wherein the disintegrant comprises low substituted hydroxypropyl cellulose, carboxymethylcellulose sodium, croscarmellose sodium, crospovidone, and sodium starch glycolate, or a mixture thereof.

22-23. (canceled)

24. The pharmaceutical composition of claim 11, wherein the lubricant comprises lauric acid, myristic acid, palmitic acid, stearic acid or pharmaceutically acceptable salts or esters thereof, such as magnesium stearate, calcium stearate, sodium stearyl fumarate, or zinc stearate or a mixture thereof.

25-27. (canceled)

28. The pharmaceutical composition of claim 11, wherein the glidant comprises talc, calcium phosphate, calcium silicate, magnesium silicate, magnesium trisilicate, silicon dioxide, colloidal silicon dioxide, magnesium aluminosilicate, or a mixture thereof.

29. (canceled)

30. The pharmaceutical composition of claim 11, wherein the composition comprises:
a) from 10 wt. % to 30 wt. % of the pretomanid,
b) from 60 wt. % to 85 wt. % of the diluent,
c) from 1 wt. % to 10 wt. % of the disintegrant,
d) from 0.1 wt. % to 1 wt. % of the surfactant,
e) from 1 wt. % to 5 wt. % of the binder,
f) from 0.1 wt. % to 1 wt. % of the glidant, and
g) from 0.1 wt. % to 3 wt. % of the lubricant.

31. The pharmaceutical composition of claim 1, wherein the composition comprises from 50 mg to 250 mg of the pretomanid.

32-33. (canceled)

34. The pharmaceutical composition of claim 1, wherein the composition is in the form of a tablet.

35. The pharmaceutical composition of claim 34, wherein the tablet has a hardness within the range of 7 to 13 Kp.

36. A method for treating tuberculosis comprising, administering to a patient in need of such treatment, a pharmaceutical composition of claim 1.

37-42. (canceled)

43. A process for preparing an oral pharmaceutical composition comprising:
preparing a granulate comprising a pharmaceutically effective amount of pretomanid and one or more pharmaceutically acceptable intragranular excipients, and
combining the granulate with one or more pharmaceutically acceptable extragranular excipients to provide a blend,
wherein the granulate has a bulk density in a range of about 0.3 to 0.8 g/ml, and
wherein the granulate has a particle size distribution such that no more than about 30 wt % of the granulate is retained on an ASTM #60 (250 μm) sieve.

44-56. (canceled)

57. The process of claim 43, wherein the particle size distribution of the granulate is such that at least 80 wt. % of the composition is retained on an ASTM #200 (75 μm) sieve.

58-59. (canceled)

60. An oral pharmaceutical composition prepared according to the process of claim 43.

61. (canceled)
